(19) United States
(12) Patent Application Publication
(10) Pub. No.: US 2024/0238510 A1
(43) Pub. Date: Jul. 18, 2024

(54) SYSTEM AND METHOD FOR CONTROLLING A TARGET CONTROLLED INFUSION

(71) Applicant: FRESENIUS Vial SAS, Brézins (FR)

(72) Inventors: Remy Wolff, Tullins (FR); Pauline Previtali, Voreppe (FR)

(21) Appl. No.: 18/561,657

(22) PCT Filed: May 3, 2022
(86) PCT No.: PCT/EP2022/061789

§ 371 Date: Nov. 16, 2023

(30) Foreign Application Priority Data
May 27, 2021 (EP) 21315090.7

(51) Int. Cl. A61M 5/142; G16H 40/63

(52) U.S. Cl. CPC A61M 5/142; G16H 40/63; A61M 2005/14208; A61M 2005/14296; A61M 2205/18; A61M 2205/502; A61M 2205/52

(57) ABSTRACT

A system for controlling a target controlled infusion for administering a drug to a patient (P) comprises at least one infusion device (31-33) for administering a drug to the patient (P) and a control device (2) configured to control operation of the at least one infusion device (31-33) in order to establish a drug concentration at an effect site within the patient (P) at or close to a target concentration. The control device (2) is configured to execute a target controlled infusion protocol using a mathematical model modelling a drug distribution in the patient's body for controlling operation of the at least one infusion device (31-33). The control device (2) is further configured to store, at a multiplicity of points in time (Tᵢ . . . Tᵢ₊₃) during execution of said target controlled infusion protocol, information derived from said mathematical model in a memory (21), to maintain at least a portion of said information in said memory (21) after a stop of execution of the target controlled infusion protocol, and to use said information in case of a start of execution of a target controlled infusion protocol after a prior stop.

The invention relates to a system for controlling a target controlled infusion for administering a drug to a patient according to the preamble of claim 1 and to a method for controlling a target controlled infusion for administering a drug to a patient.

A system of this kind comprises at least one infusion device for administering a drug to the patient and a control device configured to control operation of the at least one infusion device. The control as performed by the control device herein is such that a drug concentration at an effect site within the patient is established which is at or at least close to a target concentration, wherein the target concentration may be constant over a period of time, or may vary such that a concentration within the patient is controlled to follow a certain concentration curve. Herein, the control device is configured to execute a target controlled infusion protocol using a mathematical model modeling a drug distribution in the patient's body for controlling operation of the at least one infusion device.

“Target controlled infusion” (TCI) generally refers to an infusion operation performed by an computer-assisted infusion system which calculates a substance concentration in a particular body compartment on the basis of a mathematical model and which, after setting a target concentration, adjusts the infusion rate such that the concentration in the body compartment of the patient converges towards and is kept at the predefined target concentration. TCI infusion systems generally consist of one or multiple infusion devices and a control device, which may be separate to the infusion devices or may be integrated into an infusion device.

For setting up an infusion operation, herein, patient specific parameters such as the patient's age, weight, gender, and drug specific parameters such as the type of drug, e.g. the type of anesthetic, and a desired target concentration in a body compartment of the patient, for example relating to a drug level in the patient's brain within an anesthesia procedure, may be entered into the system using a human machine interface. In addition, a suitable mathematical model, such as a pharmacokinetic/pharmacodynamic model out of a multiplicity of models defined in the system, may be chosen for executing a target controlled infusion protocol. When performing a target controlled infusion operation, then, the control device executes the target controlled infusion protocol and in this context calculates infusion rates in order to control one or multiple infusion devices for administering one or multiple specified drugs to the patient.

On the basis of an empirically determined population-pharmacokinetic model and using a known pharmacokinetic and patient-specific pharmacodynamic parameter set of a medicament (for example propofol) as well as by means of patient-specific data, a TCI system models a drug distribution (over time) within the patient's body by calculating drug concentrations in body compartments as defined within the model. During the execution of a target controlled infusion protocol, herein, the mathematical model may be repeatedly adjusted according to measurement values relating to a drug concentration within the patient, for example by measuring a drug concentration in a patient's breath or in the patient's plasma (blood) compartment, or by measuring biological signals such as EEG or ECG signals or by deriving indices such as the so-called bi-spectral (BIS) index. According to measurement values the mathematical model is used during operation such that it suitably reflects the concentrations in the patient's body compartments according to the measurement values, such that patient-individual effects such as a patient-specific metabolism may be taken into account. The mathematical model may hence accurately model the drug concentration within the body, which may be used to control the infusion operation using one or multiple infusion devices in order to set or maintain a desired concentration in a desired effect site compartment within the patient to obtain a desired medical effect, such as an anesthetic effect during an anesthesia procedure.

Systems and methods for performing target controlled infusion operations, in particular anesthetic operations, are for example known from EP 1 418 976 B1, WO 2016/160321 A1, and WO 2017/190966 A1.

During execution of a target controlled infusion protocol it may occur that the execution suddenly is aborted, for example in case of a technical failure, by abandonment of the infusion by a user, or by undocking a pump for example from a rack at the bedside of a patient. In case of such a sudden stop of execution of the target controlled infusion protocol, the control device issues commands to participating infusion devices to stop infusion, and terminates the execution of the protocol.

When during execution of a target controlled infusion protocol one or multiple drugs have been infused into a patient in order to set a drug concentration in a body compartment of the patient according to a predefined target, the drug concentration will decay over time after abortion of the execution of the protocol, wherein a time of decay may largely depend on the type of drug, e.g. the type of anesthetic agent used during an anesthesia procedure. If subsequently another target controlled infusion operation shall be started by executing a corresponding protocol, it needs to be made sure that the prior, aborted execution of the (same or another) target controlled infusion protocol does not interfere with the new infusion operation. If prior drug concentrations in the body remaining from a prior infusion operation are significant, the new infusion operation may give rise to excessively high concentrations within the patient's body which may be hazardous to the patient. This needs to be avoided.

It is an object of the instant invention to provide a system and a method which allow for a safe start of another infusion operation by executing a target controlled infusion protocol after a prior stop of an infusion operation.

This object is achieved by means of a system comprising the features of claim 1.

Accordingly, the control device is configured to store, at a multiplicity of points in time during execution of the target controlled infusion protocol, information derived from the mathematical model in a memory, to maintain at least a portion of the information in the memory after a stop of execution of the target controlled infusion protocol, and to use the information in case of a start of execution of a target controlled infusion protocol after a prior stop.

In one embodiment, the control device is a separate device from the infusion devices.

In another embodiment, the control device is an integrated part inside the infusion devices.

The control device serves to execute a target controlled infusion protocol for performing a target controlled infusion operation. Within the target controlled infusion protocol, one or multiple drugs may be infused into a patient using one or multiple infusion devices, the infusion being such that for one or multiple drugs a drug concentration in one or multiple body compartments of a patient are caused to approach one or multiple predefined target values. During the target controlled infusion operation in particular a drug concentration of a particular drug at an effect site, for example the patient's brain, may be set to a predefined target value, such that a desired effect at the effect site is achieved, for example an anesthetic effect corresponding to a particular drug concentration.

In case a target controlled infusion operation is aborted at a certain point of time, for example due to a technical failure or due to a (terminal) stop of the infusion operation by a user command, conventionally the mathematical model is reset and information related to said mathematical model are discarded. Hence, when another infusion operation is started after some time by starting execution of the same or another target controlled infusion protocol, also the control process involving the mathematical model is started anew, wherein the mathematical model may assume a 0 drug concentration within the patient at the start, giving rise to a potentially wrong modeling of drug concentrations within the patient and potentially a drug overdose which may cause excessively high drug concentrations in the patient and which may be hazardous to the patient.

For this reason it herein is proposed to repeatedly store information related to said mathematical model during execution of the target controlled infusion protocol. In case the execution of the target controlled infusion protocol is stopped unexpectedly (intentionally or unintentionally by a user), the information is maintained in memory, preferably unchanged, such that the information relating to and derived from the mathematical model may be used when starting another target controlled infusion operation after some time.

Different types of information herein may be derived from the mathematical model and may be stored.

In one embodiment, the control device is configured to compute, at each of the multiplicity of points in time during execution of the target controlled infusion protocol, a duration based on the mathematical model and to store the duration in the memory as the information. The duration herein is indicative of a time period after lapse of which a start of execution of another (new) target controlled infusion protocol after the prior stop is admissible.

This is based on the finding that a drug concentration in one or multiple body compartments of a patient may vary over time during execution of a target controlled infusion protocol such that a decay time which is required for a drug concentration to decay towards a negligible level also varies, as the decay time depends on the actual drug concentration in the specific body compartment. Herein, it may for example be referred to an effect site within the patient, and the decay time may be computed based on the drug concentration as modeled by the mathematical model in the effect site. The duration may be computed such that it reflects such time period that is required for the drug concentration to revert to a value at or at least close to 0.

During the execution of the target controlled infusion protocol the duration is repeatedly computed and stored anew. At a time of stopping the execution, then, the last value for the duration may be kept in memory and may be used subsequently in order to assess whether another infusion operation may be started.

After lapse of the duration it may be safe to start another target controlled infusion operation by starting execution of another target controlled infusion protocol. Hence, in one embodiment the control device is configured to assess, in case of a start of execution of a (new) target controlled infusion protocol after the prior stop of execution, whether a lapsed time after a previous stop of execution of a target controlled infusion protocol is larger than said computed duration and to initiate a countermeasure in case the lapsed time is not larger than the duration. It hence is checked whether a sufficient time has elapsed between the new start of an infusion operation and the prior stop. If this is not the case, a countermeasure is initiated such that a user is warned or the start of execution of the new infusion operation is prohibited or at least delayed.

In particular, as a countermeasure a warning message may be presented (visually and/or acoustically) to a user, the warning message being produced by the control device in case it is found that the lapsed time after the previous stop of execution is not larger than the computed duration.

As another countermeasure, the control device may be configured to produce a command prohibiting the start of execution of a target controlled infusion protocol, such that no new target controlled infusion operation may be started, at least not as long as the lapsed time after the prior stop does not exceed the computed duration.

In one embodiment, the control device is configured to compute the duration to correspond to a time period that is required for a drug concentration in a patient's body compartment to fall under a predefined threshold. The threshold may be for example defined to correspond to a defined fraction of a computed concentration in a body compartment, or to a fraction of a default therapeutic drug concentration. The fraction may for example lie in a range between ⅛ to 1/64. For example, a fraction of 1/32 of a default therapeutic drug concentration may be chosen, corresponding to a level reached after 5 half-life periods of the resepctive drug.

The threshold may alternatively be programmable. For example, the threshold may be predefined as a fixed value in a drug library as stored in the memory of the system.

In one embodiment, the control device is configured to store, at each of the multiplicity of points in time during execution of the target controlled infusion protocol, a set of parameters of said mathematical model as the information in the memory and to use the set of parameters in the mathematical model in case of a start of execution of a target controlled infusion protocol after a prior stop. In this case, hence, parameters relating to the mathematical model are stored, for example concentration values and values e.g. of transfer rates for example within a pharmacokinetic/pharmacodynamic model. Hence, during execution of the target controlled infusion protocol information is repeatedly stored, e.g. providing for an actual image of the mathematical model at the different points in time. This information is maintained after a (terminal) stop of execution, and may be used when afterwards starting another infusion operation, such that for another, subsequent execution of a target controlled infusion protocol the mathematical model then used does not start fresh, but may use model parameters as previously stored during the prior execution of the target controlled infusion protocol.

When, during execution of the target controlled infusion protocol, a set of parameters relating to the mathematical model is stored, for example concentration values in different body compartments, these may not be current any longer when starting execution of another target controlled infusion protocol after some time. Hence, in one embodiment the control device is configured to compute a drug distribution in the patient's body, at the time of starting the execution of a target controlled infusion protocol after said prior stop, using the set of parameters and a lapsed time between the prior stop and the time of subsequently starting the execution of the target controlled infusion protocol. Using the lapsed time, for example a decay of the drug concentrations in the patient's body may be modeled as it has occurred during the time after stopping the prior infusion operation. The mathematical model hence is enabled to compute the current drug concentrations in the different body compartments of the patient based on the lapsed time since the prior abortion of the target controlled infusion protocol.

In this case, hence, the system starts anew with knowledge about prior conditions resulting from the abortion of the prior infusion operation. Hence, in principle no warning message to a user or no measures to potentially prohibit a new start of infusion is required, but the execution of the target controlled infusion operation may be started anew using the set of parameters as stored previously and as valid at the time of abortion of the prior infusion operation.

In one embodiment, the control device is configured to associate the information as stored in memory with a timestamp indicative of a corresponding of said multiplicity of points in time, and to store the information together with the associated timestamp in the memory. The information as derived from the mathematical model hence is timestamped, such that, after aborting execution of the target controlled infusion protocol, most recent information can be used when subsequently starting execution of another target controlled infusion protocol.

In one embodiment, the control device is configured to update the information in the memory by overwriting the information in the memory stored at a point in time by updated information computed at a subsequent point in time. Hence, information is repeatedly derived from the mathematical model, for example by computing a duration or by storing a set of parameters relating to the mathematical model, wherein not all information is maintained over all times, but current information is used to overwrite prior information, such that in case of abortion of an infusion operation (only) the most recent information is available and can be used in a subsequent start of execution of a target controlled infusion protocol.

In one embodiment, the multiplicity of points in time are equidistantly spaced apart at a predefined time interval. Hence, at regular intervals information is derived and stored, the interval being chosen such that suitable information is available at any time, independent from the actual time of abortion of execution of the target controlled infusion operation.

In another embodiment, information may be derived and stored in an event-driven manner, for example at every time that substantial changes in the mathematical model or a drug concentration within the patient's body occur during execution of the target controlled infusion protocol.

The mathematical model in particular may be a pharmacokinetic/pharmacodynamic model which models the drug distribution of a drug administered to a patient. Within the pharmacokinetic/pharmacodynamic model the drug concentration is modeled in different body compartments of a patient, in particular a plasma compartment, a brain compartment, a rapid equilibrating compartment (representative e.g. of muscle and inner organ tissue) and a slow equilibrating compartment (e.g. fat, bone tissue). The model herein may self-adjust during execution of the target controlled infusion protocol in dependence on measurement values as obtained during execution, such that the model is individualized during execution and hence reflects patient-specific conditions as experienced during the target controlled infusion operation.

In another aspect, a method for controlling a target controlled infusion for administering a drug to a patient comprises: controlling, using a control device, operation of at least one infusion device in order to establish a drug concentration at an effect site within the patient at or close to a target concentration by executing a target controlled infusion protocol using a mathematical model modelling a drug distribution in the patient's body for controlling operation of the at least one infusion device; storing, by the control device, information derived from said mathematical model in a memory at a multiplicity of points in time during execution of said target controlled infusion protocol; maintaining, by the control device, at least a portion of said information in said memory after a stop of the execution of the target controlled infusion protocol; and using, by the control device, said information in case of a start of execution of a target controlled infusion protocol after a prior stop.

The advantages and advantageous embodiments as described above for the system equally apply also to the method, such that it shall be referred to the above in this respect.

The idea underlying the invention shall subsequently be described in more detail by referring to the embodiments shown in the figures. Herein:

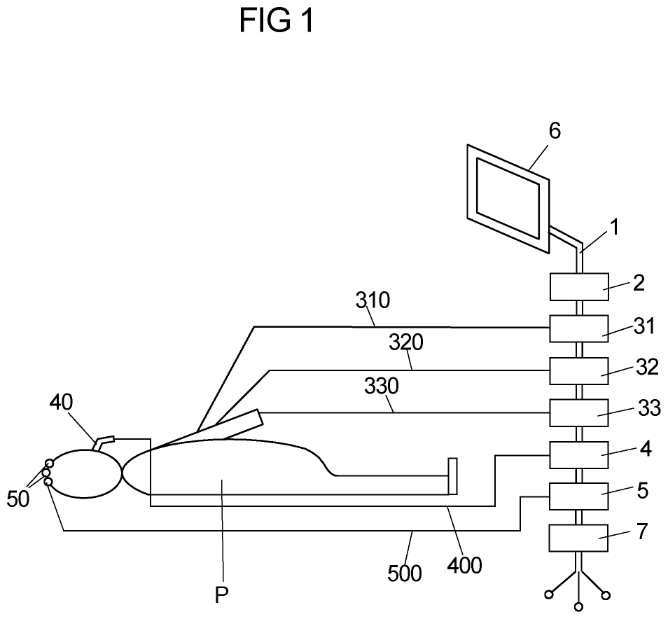

FIG. 1 shows a schematic view of a setup of system for performing a target controlled infusion (TCI);

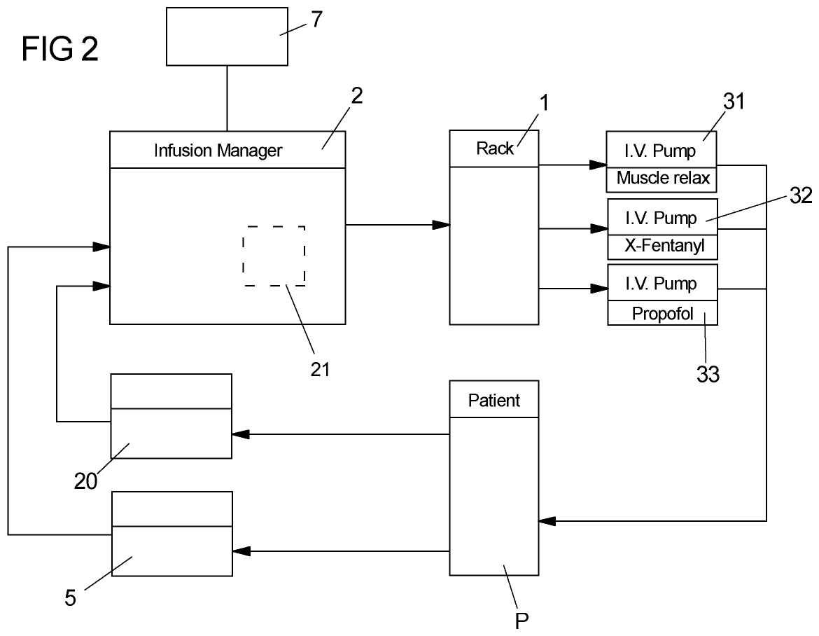

FIG. 2 shows a functional diagram of the setup of FIG. 1;

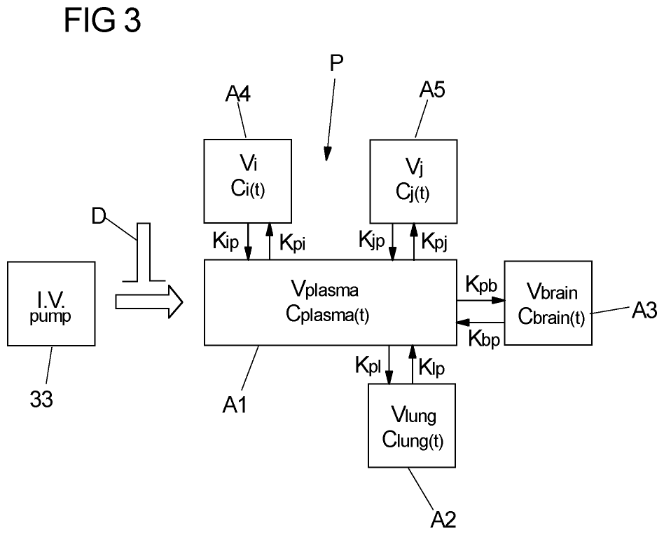

FIG. 3 shows a functional diagram of a model for modelling the distribution of a drug dosage in a patient's body;

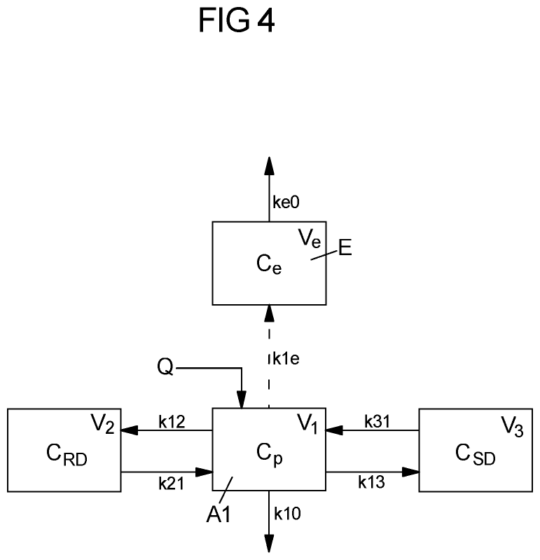

FIG. 4 shows a schematic diagram of a PK/PD model;

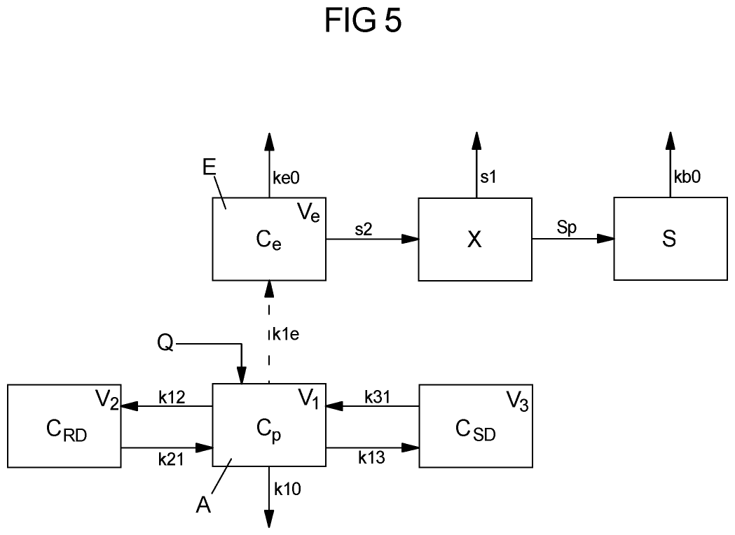

FIG. 5 shows a schematic diagram of another PK/PD model;

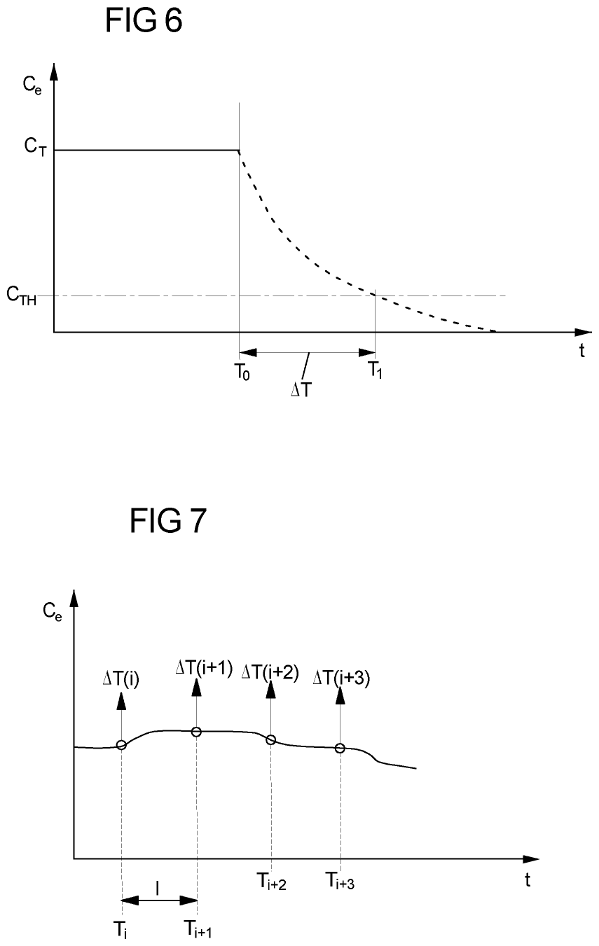

FIG. 6 shows a schematic concentration curve as a function of time after stopping an infusion operation;

FIG. 7 shows a concentration curve during an infusion operation, indicating the deriving of duration information at particular intervals during the infusion operation; and

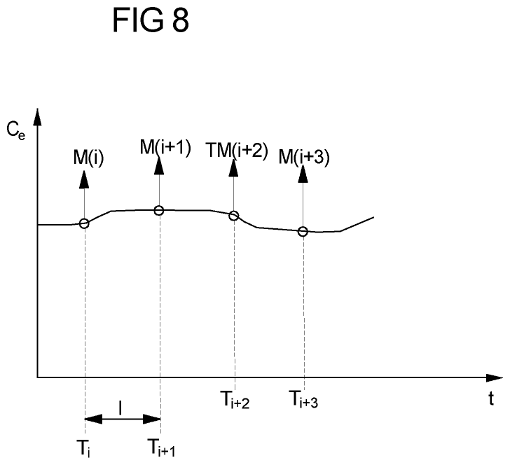

FIG. 8 shows a concentration curve, indicating the deriving of information relating to a mathematical model during the infusion operation.

Subsequently, a system and method for administering one or multiple drugs to a patient in a target controlled infusion (TCI) procedure, e.g. an anesthetic procedure, shall be described in certain embodiments. The embodiments described herein shall not be construed as limiting for the scope of the invention.

Like reference numerals are used throughout the figures as appropriate.

FIG. 1 shows a schematic drawing of a setup as it generally is used for example in an anesthesia procedure for administering anesthetic drugs, such as analgesic agents or hypnotic agents, e.g. propofol and/or remifentanil, to a patient P. In this setup multiple devices are arranged on a rack 1 and are connected via different lines to the patient P.

In particular, infusion devices 31, 32, 33 such as infusion pumps, in particular syringe pumps or volumetric pumps, are connected to the patient P and serve to intravenously inject, via lines 310, 320, 330, different drugs such as propofol, remifentanil and/or a muscle relaxant drug to the patient P in order to achieve a desired anesthetic effect. The lines 310, 320, 330 are for example connected to a single port providing access to the venous system of the patient P such that via the lines 310, 320, 330 the respective drugs can be injected into the patient's venous system.

The rack 1 furthermore may hold a ventilation device 4 for providing an artificial respiration to the patient P e.g. while the patient P is under anesthesia. The ventilation device 4 is connected via a line 400 to a mouth piece 40 such that it is in connection with the respiratory system of the patient P.

The rack 1 also holds a bio-signal monitor 5, for example an EEG monitor which is connected via a line or a bundle of lines 500 to electrodes 50 attached to the patient's head for monitoring the patient's brain activity e.g. during an anesthesia procedure.

In addition, a control device 2 is held by the rack 1 which serves to control the infusion operation of one or multiple of the infusion devices 31, 32, 33 such that infusion devices 31, 32, 33 inject drugs to the patient P in a controlled fashion to obtain a desired effect, e.g. an anesthetic effect. This shall be explained in more detail below.

Additional measurement devices may be used, e.g. for measuring the concentration of one or multiple drugs for example in the breath of the patient P or to measure information relating to and allowing to determine e.g. a bi-spectral index. A measurement device may for example be constituted by a so called IMS monitor for measuring a drug concentration in the patient's breath by means of the so called Ion Mobility Spectrometry. Other sensor technologies may also be used.

FIG. 2 shows a functional diagram of a control loop for controlling the infusion operation of infusion devices 31, 32, 33 during an infusion operation. The control loop herein may in principle be set up as a closed-loop in which the operation of the infusion devices 31, 32, 33 is automatically controlled without user interaction. Alternatively, the system is set up as an advisory (open-loop) system in which at certain points of time, in particular prior to administering a drug dosage to a patient, a user interaction is required in order to manually confirm the operation.

The control device 2, also denoted as “infusion manager”, is connected to the rack 1 which serves as a communication link to the infusion devices 31, 32, 33 also attached to the rack 1. The control device 2 outputs control signals to control the operation of the infusion devices 31, 32, 33, which according to the received control signals inject defined dosages of drugs to the patient P.

By means of the bio-signal monitor 5, e.g. in the shape of an EEG monitor, for example an EEG reading of the patient P is taken, and by another measurement device 20 a concentration of one or multiple drugs in the patient's P breath is measured. The measured data are fed back to the control device 2, which correspondingly adjusts its control operation and outputs modified control signals to the infusion devices 31, 32, 33 to achieve a desired anesthetic effect.

The control device 2 uses, for controlling the infusion operation of one or multiple infusion devices 31, 32, 33, a pharmacokinetic-pharmacodynamic (PK/PD) model, which is a pharmacological model for modelling processes acting on a drug in the patient's P body. Such processes include the resorption, the distribution, the biochemical metabolism and the excretion of the drug in the patient's P body (denoted as pharmacokinetics) as well as the effects of a drug in an organism (denoted as pharmacodynamics). Preferably, a physiological PK/PD model with N compartments is used for which the transfer rate coefficients have been experimentally measured beforehand (for example in a proband study) and are hence known.

A schematic functional drawing of the setup of such a PK/PD model p is shown in FIG. 3. The PK/PD model p logically divides the patient P into different compartments A1-A5, for example a plasma compartment A1 corresponding to the patient's P blood stream, a lung compartment A2 corresponding to the patient's P lung, a brain compartment A3 corresponding to the patient's P brain and other compartments A4, A5 corresponding, for example, to muscular tissue or fat and connective tissue. The PK/PD model p takes into account the volume V_{Lung}, Vₚₗₐₛₘₐ, V_{brain}, Vᵢ, Vⱼ of the different compartments A1-A5 as well as transfer rate constants K_{PL}, K_{LP}, K_{BP}, K_{PB}, K_{IP}, K_{PI}, K_{JP}, K_{PJ} indicating the transfer rates between the plasma compartment A1 and the other compartments A2-A5, assuming that a drug dosage D by means on an infusion device 31-33 is injected into the plasma compartment A1 and the plasma compartment A1 links the other compartments A2-A5 such that an exchange between the other compartments A2-A5 always takes place via the plasma compartment A1. The PK/PD model p serves to predict the concentrations C_{lung}, Cₚₗₐₛₘₐ, C_{brain}, Cᵢ, Cⱼ of the injected drug in the different compartments A1-A5 as a function of time.

FIG. 4 illustrates, in a schematic diagram, an example of a PK/PD model which comprises a central plasma compartment A1 exhibiting a drug concentration Cₚ, a rapid equilibrating compartment exhibiting a drug concentration C_{RD}, a slow equilibrating compartment exhibiting a drug concentration C_{SD}, an dan effect compartment E comprising an effect compartment concentration Cₑ of the drug. Herein,
Q represents an administered drug,
kₑ₀ defines the proportional change in each unit of time of the concentration gradient between the plasma and effect-site,
k₁ₑ describes an elimination constant for redistribution of the drug from the effect compartment E to the plasma compartment A1,
k₁₂ is an elimination constant describing the distribution of the volume V1 in direction of volume V2,
k₂₁ is an elimination constant describing the distribution of the volume V2 in direction of volume V1,
k₁₃ is an elimination constant describing the distribution of the volume V1 in direction of volume V3,
k₃₁ is an elimination constant describing the distribution of the volume V3 in direction of volume V1,
k₁₀ represents the elimination constant of the applied drug from the body.

FIG. 4 herein visualizes a so called Schnider model. This assumes that after intravenous injection a drug Q is rapidly distributed in the circulation of the central plasma compartment A1 and quickly reaches well perfused tissues, such that a tissue-specific redistribution in various other compartments such as muscle or fat tissue occurs. At the same time the body eliminates the applied substance from the plasma compartment A1 with a certain elimination rate. For the pharmacokinetic characterization e.g. of lipophilic anesthetics, a 3-compartment model has been established that comprises a plasma compartment A1 (heart, lung, kidney, brain), a rapid equilibrating compartment exhibiting a concentration C_{RD} (muscles, inner organs), and a slow equilibrating compartment exhibiting a concentration C_{SD} (fat, bone, the so-called “deep” compartment). The concentration-time curve of a drug is characterized by the distribution volume of a specific compartment and the clearance (which is the plasma volume, from which the drug is eliminated per time unit): V1 denotes the volume of the plasma compartment A1, V2 is the volume of a well-perfused tissue C_{RD} and V3 is the volume of a less perfused compartment, associated with a concentration C_{SD}. The clearance of a substance from the various compartments can be described by elimination constants. The elimination constant k₁₂ for example describes the distribution from the volume V1 towards the volume V2, and k₂₁ describes the distribution in the opposite direction. An applied substance is eliminated by this model with the constant k₁₀ from the body. After reaching an equilibrium (“steady state”) between the individual compartments, the elimination rate determines the amount of substance that must be supplied to maintain equilibrium.

To assess the clinical effect (the so-called pharmacodynamics) of a drug at the target site, dose-response curves are generally used. Such curves, which are typically of a sigmoidal shape, describe the association between the drug concentration and a particular clinical effect. Knowing the dose-response relationship, a putative drug concentration at the site of action, the effect compartment E, can be calculated.

FIG. 5 shows a schematic diagram of another example of a PK/PD model, as it for example is described in WO 2017/190966 A1. In comparison to the model of FIG. 4, the model additionally comprises a remote compartment X and a BIS sensor compartment S, wherein
s1 and s2 represent constant transfer rate parameters between the remote compartment X and the effect compartment E,
S_{P} represents a transfer rate coefficient between the remote compartment X and the BIS sensor S, and
k_{b0} represents the decay rate of the BIS index.

Clinically, S_{P} can be regarded as a sensitivity value. The higher the value of S_{P}, the faster the drug's effect is achieved. High values of S_{P} further lead to a small delay and a high responsiveness of the system.

The remote compartment X describes the delay between the drug's concentration in the effect-site compartment and its actual impact on the BIS value.

TCI models, e.g. for propofol, are known in the art. Recently introduced open-target controlled infusion (TCI) systems can be programmed with any pharmacokinetic model, and allow either plasma- or effect-site targeting. With effect-site targeting the goal is to achieve a user-defined target effect-site concentration as rapidly as possible, by manipulating the plasma concentration around the target. Currently systems are for example pre-programmed with a Marsh model (B. Marsh et al., “Pharmacokinetic model driven infusion of propofol in children” Br J Anaesth, 1991; 67, pages 41-48) or a Schnider model (Thomas W. Schnider et al., “The influence of method of administration and covariates on the pharmacokinetics of propofol in adult volunteers”, Anesthesiology, 1998, 88(5) pages 1170-82).

The PK/PD model as shown in FIG. 5 can mathematically be described by the following set of (differential) equations.

Namely, the S compartment is described according to:

$$\dot{S} = \frac{{s}_{p} X}{{α}_{M} + X} - {k}_{b 0} S + O F \qquad ( Equation 1 )$$

wherein
s_{P} represents the drug sensitivity of the patient;
α_{M} represents the saturation parameter of the velocity of effect of a drug, e.g. an anesthetic agent such as propofol (i.e. the saturation of the drug receptors);
k_{b0} represents the decay rate of the BIS index;
OF represents the offset that can remain when no more drug is present in the patient body;
X represents a remote compartment; and
S represents a sensor value of a BIS sensor.

The X compartment is described by:

$$\dot{X} = {s}_{2} {C}_{e} - {s}_{1} X . \qquad ( Equation 2 )$$

wherein
s₁ and s₂ represent constant transfer rate parameters between the remote compartment and the effect parameters;
Cₑ represents the effect compartment concentration; and
X represents a remote compartment.

The rapid equilibrating compartment C_{RD} is described by:

$${\dot{C}}_{RD} = - {k}_{21} {C}_{RD} + {k}_{12} {C}_{p} \qquad ( Equation 3 )$$

wherein
k₁₂ is an elimination constant describing the drug distribution from the plasma compartment A1 in direction of rapid equilibrating compartment C_{RD},
k₂₁ is an elimination constant describing the drug distribution from rapid equilibrating compartment C_{RD} in direction of plasma compartment A1,
C_{RD} represents the concentration in the rapid equilibrating compartment, and
Cₚ represents the drug concentration in the plasma (blood) compartment.

The slow equilibrating compartment C_{SD} is described by:

$${\dot{C}}_{SD} = - {k}_{31} {C}_{SD} + {k}_{13} {C}_{p} \qquad ( Equation 4 )$$

wherein
k₁₃ is an elimination constant describing the drug distribution from plasma compartment A1 in direction of slow equilibrating compartment C_{SD},
k₃₁ is an elimination constant describing the drug distribution from slow equilibrating compartment C_{SD} in direction of plasma compartment A1,
C_{SD} represents the concentration in the slow equilibrating compartment; and
Cₚ represents the drug concentration in the plasma (blood) compartment.

The effect compartment concentration Cₑ is described by:

$${\dot{C}}_{e} = - {k}_{e 0} {C}_{e} + {k}_{1 e} {C}_{p} \qquad ( Equation 5 )$$

wherein
kₑ₀ defines a decay rate;
k₁₀ describes a “virtual” constant rate transfer from plasma compartment A1 and the effect compartment E; and
Cₑ represents the effect comportment concentration.

The blood concentration Cₚ is described by:

$${\dot{C}}_{p} = - ( {k}_{10} + {k}_{12} + {k}_{13} ) {C}_{p} + {k}_{21} {C}_{RD} + {k}_{31} {C}_{SD} \qquad ( Equation 6 )$$

wherein
k₁₀ represents the elimination constant of an applied drug from the body,
k₁₂ is an elimination constant describing the drug distribution from the plasma compartment A1 in direction of rapid equilibrating compartment C_{RD},
k₂₁ is an elimination constant describing the drug distribution from rapid equilibrating compartment C_{RD} in direction of plasma compartment A1,
k₁₃ is an elimination constant describing the drug distribution from plasma compartment A1 in direction of slow equilibrating compartment C_{SD},
k₃₁ is an elimination constant describing the drug distribution from slow equilibrating compartment C_{SD} in direction of plasma compartment A1,
C_{RD} represents a rapid equilibrating compartment;
C_{SD} represents a slow equilibrating compartment; and
Cₚ represents the drug concentration in the plasma (blood) compartment.

Generally, the mathematical model, e.g. a PK/PD model as described above, during execution of an infusion operation is used to model the drug concentrations in certain body compartments of the patient, such that information about the drug distribution during the infusion operation is available for controlling the infusion operation using one or multiple infusion devices. The control herein is such that at the effect site, for example in the patient's brain, a drug concentration is established which is at or close to a desired target concentration, wherein for this the control device 2 (FIGS. 1 and 2) controls infusion devices 31 to 33 such that a drug is infused to reach and maintain a drug concentration at the effect site at or close to the desired target concentration.

During execution of the infusion operation, the mathematical model may be tuned according to measurement values as obtained for example by a bio-signal monitor or from a sensor for measuring a drug concentration in the exhaled breath of a patient or the like. Using measurement information the mathematical model may be tuned according to actual concentration information by adjusting parameters of the model, for example transfer rate parameters or the like, such that the model correctly reflects the measured information and hence reliably predicts the drug concentrations in the different body compartments.

Referring now to FIG. 6, during an infusion operation using a target controlled infusion protocol a drug concentration Cₑ at an effect site within the patient, for example within the patient's brain, may be held in equilibrium at a drug concentration corresponding to a target concentration C_{T}. If, at time T₀, the execution of the target controlled infusion protocol is stopped, for example due to a technical error, due to an intentional abortion of the infusion operation or by disconnecting an infusion device for example from a rack at the bedside of a patient, no drug is infused to the patient any longer, such that the drug concentration will generally decay towards 0 starting at the time of abortion T₀.

Herein, it will take a substantial time until the drug concentration at the effect site has decayed to a level which is negligible, i.e., that is smaller than a threshold value C_{TH}. If, soon after stopping the infusion operation at time T₀, another infusion operation is started by starting execution of another target controlled infusion protocol, it may be the case that one or multiple drugs are infused to the patient without the knowledge about a prior infusion operation and without knowledge about actual, remaining drug concentrations within the patient due to the prior infusion operation.

This is because conventionally, in case of an abortion of an infusion operation by (terminally) stopping the execution of a target controlled infusion protocol, all information with respect to the execution are deleted, because the system is reset, such that upon a subsequent start of execution of another target controlled infusion protocol the mathematical model is started anew and assumes a start at 0 conditions, hence assuming no drugs to be present within the patient at the start of the infusion operation.

For this reason it is proposed to repeatedly store information derived from the mathematical model during execution of the target controlled infusion protocol, and to keep the information in a memory 21 (see FIG. 2) of the control device 2 even in case the execution of the target controlled infusion protocol is unexpectedly stopped.

The information may for example be a duration ΔT which is repeatedly computed during execution of a target controlled infusion protocol and reflects that time period that is required for the drug concentration Cₑ at the effect site compartment to fall under a predefined concentration threshold C_{TH}, as it is illustrated in FIG. 6.

Referring now to FIG. 7, during execution of a target controlled infusion protocol, the duration is repeatedly computed anew at points in time Tᵢ . . . Tᵢ₊₃, wherein the times point in time Tᵢ . . . Tᵢ₊₃ may be equidistantly spaced at time intervals I. At each point in time Tᵢ . . . Tᵢ₊₃ a value for the duration T(i) . . . T(i+3) is computed and stored in the memory 21, wherein a current value may overwrite a prior value such that the duration value is continuously updated.

If now, at the time T₀, as illustrated in FIG. 6, an infusion operation is (intentionally or unintentionally) terminally stopped, the value for the duration is maintained. If, subsequently, another infusion operation shall be started by executing the target controlled infusion protocol anew, it is checked whether the lapsed time between the time of the new start and the prior stop at time T₀ exceeds the stored duration, and only if this is the case the start of the new infusion operation is admitted without further ado. If this is not the case, a suitable countermeasure may be initiated, for example by producing a warning message to a user, or by producing a command prohibiting the start of the new infusion operation.

The duration is repeatedly computed anew during the ongoing execution of the target controlled infusion protocol. Depending on the actual, current concentration Cₑ at the effect site, the duration herein may vary, the duration indicating the time that is required for the concentration to fall beneath the predefined threshold C_{TH}.

The threshold C_{TH} herein may be determined to correspond to a fraction of a default therapeutic drug concentration at the effect site, to a fraction of the actual, current concentration Cₑ at the specific point in time, or may be a fixed value, which is for example programmed in a drug library. For example, the threshold concentration C_{TH} may be set to a fraction of 1/32 of a default therapeutic drug concentration, hence indicating a time that matches a decay within 5 half-life periods of a drug.

The computed duration generally depends on the drug and is computed using a decay rate as it is defined for a particular drug within the model (see, e.g., equation 5 above, decay rate kₑ₀).

Referring now to FIG. 8, in another example a set of parameters relating to the mathematical model may be stored at different points in time Tᵢ . . . Tᵢ₊₃. The set of parameters M(i) . . . M(i+3) may represent all parameters as shown above in the system of equations representing the mathematical model, that is the computed drug concentrations in the different body compartments, the transfer rate constants, the decay rate constants and the like at the different points in time Tᵢ . . . Tᵢ₊₃.

Herein, at a point in time Tᵢ . . . Tᵢ₊₃ the corresponding, current set of parameters M(i) . . . M(i+3) may be used to overwrite a previous set of parameters, such that only the most recent set of parameters is kept in storage.

At the different points in time Tᵢ . . . Tᵢ₊₃, hence, a snapshot of the model is stored in memory 21, and is kept even if at the time T₀ execution of the target controlled infusion protocol is suddenly aborted. If, after some time, execution of the target controlled infusion protocol shall be started anew, the previously stored set of parameters M(i+3) may be used for the new execution of the target controlled infusion protocol, such that the mathematical model is initialized with the previously stored information.

Hence, during the new execution of the target controlled infusion protocol actual drug concentrations as resulting from the previous infusion operation may be computed and taken into account, such that an over-dosage during the new execution of the target controlled infusion protocol is avoided.

The information as derived from the mathematical model during execution of the target controlled infusion protocol generally is timestamped, such that the information is stored in memory together with an associated timestamp indicating the associated point in time Tᵢ . . . Tᵢ₊₃. This allows, for example, to determine an elapsed time between the stored, prior information and a new start of the execution of the target controlled infusion protocol, such that based on the lapsed time e.g. current drug concentrations at the new start of execution of the target controlled infusion protocol may be computed.

The idea of the invention is not limited to the embodiments described above, but may be implemented in a different fashion.

A target controlled infusion may generally be used for performing an anesthesia operation on a patient, but may also be employed for infusing drugs to a patient to achieve a therapeutic action.

An infusion operation herein may involve one or multiple drugs administered using one or multiple infusion devices

### LIST OF REFERENCE NUMERALS

1 Rack
2 Control device
20 Measurement device
21 Memory
31, 32, 33 Infusion device
310, 320, 330 Line
4 Ventilation device
40 Mouth piece
400 Line
5 Bio-signal monitor
50 Electrodes
500 Line
6 Display device
7 Monitor device
A1-A5 Compartments
Cₑ Effect site concentration
Cp Concentration of plasma compartment
C_{RD} Concentration of rapid equilibrating compartment
C_{SD} Concentration of slow equilibrating compartment
C_{T} Target concentration
C_{TH} Threshold concentration
D Drug dosage
ΔT Duration
E Effect site compartment
I Interval
k12, k21, k31, k13, k1e, k10 Parameters
kb0 Decay rate
P Patient
S Sensor value
s1, s2 Transfer rate parameter
T0, T1 Point in time
Tᵢ . . . Tᵢ₊₃ Point in time
Q Drug
U Operator (practioner)
V₁-V₃, Vₑ Volume
X Remote compartment

1. A system for controlling a target controlled infusion for administering a drug to a patient, comprising:
at least one infusion device for administering a drug to the patient; and a control device configured to control operation of the at least one infusion device in order to establish a drug concentration at an effect site within the patient at or close to a target concentration;
wherein the control device is configured to execute a target controlled infusion protocol using a mathematical model modelling a drug distribution in the patient's body for controlling operation of the at least one infusion device;
wherein the control device is configured to store, at a multiplicity of points in time (Tᵢ . . . Tᵢ₊₃) during execution of said target controlled infusion protocol, information derived from said mathematical model in a memory, to maintain at least a portion of said information in said memory after a stop of execution of the target controlled infusion protocol, and to use said information in case of a start of execution of a target controlled infusion protocol after a prior stop.

2. The system according to claim 1, wherein the control device is configured to maintain, during a time period in which the execution of the target controlled infusion protocol is stopped, said information in said memory unchanged.

3. The system according to claim 1, wherein the control device is configured to compute, at each of the multiplicity of points in time (Tᵢ . . . Tᵢ₊₃) during execution of the target controlled infusion protocol, a duration (ΔT) based on said mathematical model and to store the duration (ΔT) in said memory as said information, wherein the duration (ΔT) is indicative of a time period after lapse of which a start of execution of a target controlled infusion protocol after said prior stop is admissible.

4. The system according to claim 3, wherein the control device is configured to assess, in case of a start of execution of a target controlled infusion protocol after said prior stop, whether a lapsed time after a previous stop of execution of the target controlled infusion protocol is larger than said duration (ΔT) and to initiate a countermeasure in case the lapsed time is not larger than said duration (ΔT).

5. The system according to claim 4, wherein the control device is configured to produce, as said countermeasure, a warning message to be displayed to a user or a command prohibiting the start of execution of a target controlled infusion protocol.

6. The system according to claim 3, wherein the control device is configured to compute said duration (ΔT) to correspond to a time period that is required for a drug concentration (Cₑ) in a patient's body compartment to fall under a predefined threshold (C_{TH}).

7. The system according to claim 1 wherein the control device is configured to store, at each of the multiplicity of points in time (Tᵢ . . . Tᵢ₊₃) during execution of the target controlled infusion protocol, a set of parameters of said mathematical model as said information in said memory and to use that set of parameters in the mathematical model in case of a start of execution of a target controlled infusion protocol after said prior stop.

8. The system according to claim 7, wherein the control device is configured to compute a drug distribution in the patient's body, at the time of starting the execution of a target controlled infusion protocol after said prior stop, using the set of parameters and a lapsed time between said prior stop and the time of subsequently starting the execution of the target controlled infusion protocol.

9. The system according to claim 1, wherein the control device is configured to associate said information with a timestamp indicative of a corresponding of said multiplicity of points in time, and to store said information together with the associated timestamp in the memory.

10. The system according to claim 1, wherein the control device is configured to update said information by overwriting the information in the memory stored at a point in time by updated information computed at a subsequent point in time.

11. The system according to claim 1, wherein the multiplicity of points in time (Tᵢ . . . Tᵢ₊₃) are equidistantly spaced apart at predefined time intervals (I).

12. The system according to claim 1, wherein the mathematical model is a
pharmacokinetic/pharmacodynamic model.

13. The system according to claim 1, wherein the mathematical model models a drug concentration in a multiplicity of compartments of the patient during execution of the target controlled infusion protocol.

14. The system according to claim 13, wherein the mathematical model is described by a multiplicity of parameters, wherein the control device is configured to adjust at least a subgroup of said multiplicity of parameters during execution of the target controlled infusion protocol according to measurement values relating to a drug concentration distribution in the patient.

15. A method for controlling a target controlled infusion for administering a drug to a patient, comprising:
controlling, using a control device, operation of at least one infusion device in order to establish a drug concentration at an effect site within the patient at or close to a target concentration by executing a target controlled infusion protocol using a mathematical model modelling a drug distribution in the patient's body for controlling operation of the at least one infusion device;
storing, by the control device, information derived from said mathematical model in a memory at a multiplicity of points in time (Tᵢ . . . Tᵢ₊₃) during execution of said target controlled infusion protocol;
maintaining, by the control device, at least a portion of said information in said memory after a stop of the execution of the target controlled infusion protocol; and
using, by the control device, said information in case of a start of execution of a target controlled infusion protocol after a prior stop.
